(19) United States
(12) Patent Application Publication
(10) Pub. No.: US 2018/0318197 A1
(43) Pub. Date: Nov. 8, 2018

(54) SOYASAPONIN-CONTAINING COMPOSITION FOR SKIN WHITENING

(71) Applicant: AMOREPACIFIC CORPORATION, Seoul (KR)

(72) Inventors: Jeong Ah HWANG, Yongin-si, Gyeonggi-do (KR); Young Gyu KANG, Yongin-si, Gyeonggi-do (KR); Jae Young KO, Yongin-si, Gyeonggi-do (KR); Hae Kwang LEE, Yongin-si, Gyeonggi-do (KR); Jun Seong PARK, Yongin-si, Gyeonggi-do (KR)

(21) Appl. No.: 15/772,364

(22) PCT Filed: Oct. 27, 2016
(86) PCT No.: PCT/KR2016/012158

§ 371 Date: Apr. 30, 2018

(30) Foreign Application Priority Data
Oct. 30, 2015 (KR) 10-2015-0152272

(51) Int. Cl. A61K 8/60; A61Q 19/02

(52) U.S. Cl. CPC A61K 8/602; A61Q 19/02

(57) ABSTRACT

The present invention relates to a composition for skin whitening containing soyasaponin or a derivative thereof, and more particularly, to a composition which is very safe to the skin with no side effects by using, as an active ingredient, a soyasaponin which can be extracted from a plant, exhibits a significantly excellent whitening effect by inhibiting skin pigmentation by inhibiting melanin production in the skin, reduces pigmentation symptoms such as melasma and freckles or improves skin dullness and enables an even and bright skin tone.

### PRIORITY CLAIM

This application is the U.S. national phase of International Application No. PCT/KR2016/012158 filed 27 Oct. 2016, which designated the U.S. and claims priority to KR Patent Application No. 10-2015-0152272 filed 30 Oct. 2015, the entire contents of each of which are hereby incorporated by reference.

### TECHNICAL FIELD

The present invention relates to a composition for skin whitening containing soyasaponin or a derivative thereof, and more particularly, to a composition which is very safe to the skin with no side effects by using, as an active ingredient, a soyasaponin which can be extracted from a plant, exhibits a significantly excellent whitening effect by inhibiting skin pigmentation by inhibiting melanin production in the skin, reduces pigmentation symptoms such as melasma and freckles or improves skin dullness and enables an even and bright skin tone.

### BACKGROUND ART

Human skin color is determined by several factors including the activity of melanocytes that produce melanin pigment, the distribution of blood vessels, the thickness of skin, and the presence or absence of pigments inside and outside the human body, such as carotenoids and bilirubin. In particular, black pigment called melanin produced by the action of several enzymes such as tyrosinase in melanocyte is the most important factor. Genetic factors, physiological factors associated with hormone secretion, stress, etc. and environmental factors such as ultraviolet irradiation affect the formation of melanin pigment. Melanin exists in the skin and has an important function of protecting the body from ultraviolet rays and the like. However, excessively produced melanin is known to accelerate pigmentation and skin aging and to induce skin cancer. In order to treat or ameliorate skin pigmentation abnormalities or excessive melanin pigmentation caused by exposure to UV or the like, ascorbic acid, kojic acid, arbutin, hydroquinone, glutathione or derivatives thereof, substances having inhibitory activity against tyrosinase have been added to cosmetics or medicines. However, their use is restricted because of insufficient skin whitening effect, skin safety issue, and stability issue within formulations when added to cosmetics.

### PRIOR ART DOCUMENT

### Patent Document

1. Korean Patent Laid-open Publication No. 10-2012-0083943 (published on Jul. 27, 2012)

### DETAILED DESCRIPTION OF THE INVENTION

### Technical Problem

Thus, the present inventors have conducted intensive studies to find out substances exhibiting excellent skin whitening effect, and found that soyasaponin present in soybean can provide excellent skin whitening effect, while having less skin irritation, thereby completing the present invention.

Therefore, an object of the present invention is to provide a composition exhibiting excellent skin whitening effect by using soyasaponin.

### Technical Solution

In order to achieve the above object, the present invention provides an external use skin preparation composition for skin-whitening or a pharmaceutical composition comprising soyasaponin or a derivative thereof as an active ingredient.

The present invention also provides use of soyasaponin or a derivative thereof as a skin whitening agent in the preparation of an external use skin preparation composition (specifically, a cosmetic composition or a pharmaceutical composition).

### Advantageous Effects

The soyasaponin used in the present invention is a natural compound present in plants and is used as medicinal herbs. Thus, it can be applied to the skin without side effects and can provide excellent skin whitening effect, reduce pigmentation symptoms such as melasma and freckles, or improve skin dullness, and enables an even and bright skin tone.

### BRIEF DESCRIPTION OF DRAWINGS

FIG. 1 shows the structure of group A soyasaponin.

FIG. 2 shows group B soyasaponin, group E soyasaponin, and group DDMP soyasaponin.

FIG. 3 shows the degree of intracellular melanin synthesis increased by α-MSH.

FIG. 4 shows the degree of extracellular melanin synthesis increased by α-MSH.

FIG. 5 shows the result of measuring the tyrosinase inhibitory activity of soyasaponin Aa using the method for evaluating the cell extract tyrosinase inhibitory activity.

### DETAILED DESCRIPTION OF THE EMBODIMENTS

The present invention relates to a composition for skin whitening, comprising soyasaponin or a derivative thereof as an active ingredient.

Saponins are a glycoside compound found in plant species and have a structure in which at least one saccharide molecule is bound to aglycone such as steroid or triterpenoid, and it is known that about 500 kinds of plants contain saponin. Many kinds of saponins are present even in soybeans, and soyasaponin, which is a saponin of soybean, has a complex and diverse molecular structure based on the aglycone structure. Specifically, soyasaponin is largely classified into group A soyasaponin, group B soyasaponin, and group E soyasaponin according to soyasapogenol A represented by Chemical Formula 1 below, soyasapogenol B represented by Chemical Formula 2 below, and soyasapogenol E represented by Chemical Formula 3 below, respectively. The group A soyasaponin is a bisdesmoside saponin in which saccharide chains are bound at two positions of C-3 and C-22, whereas the group B soyasaponin and group E soyasaponin are monodesmoside saponins in which a saccharide chain is bound at one position of C-3. Further, the group B soyasaponin is divided into the one in which DDMP (2,3-dihydro-2,5-dihydroxy-6-methyl-4H-pyran-4-one) is bound at the C-22 position, and the one in which no DDMP is bound. Among them, the one in which DDMP is bound may be classified as a group DDMP soyasaponin apart from the group B soyasaponin.

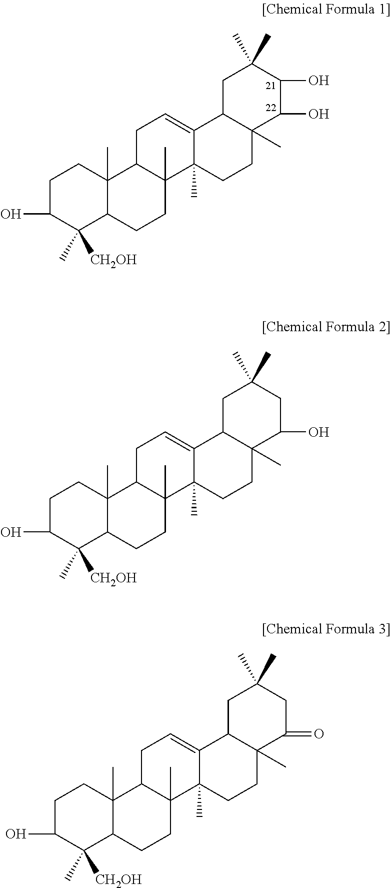

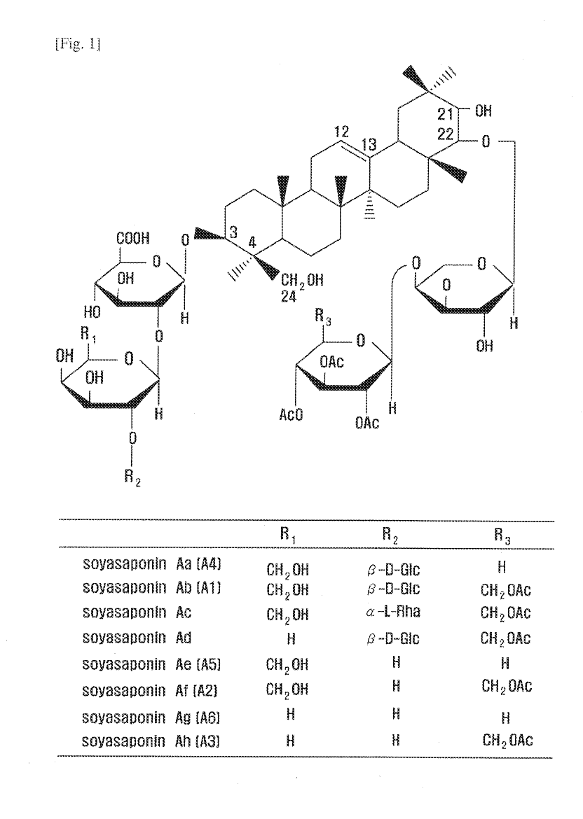

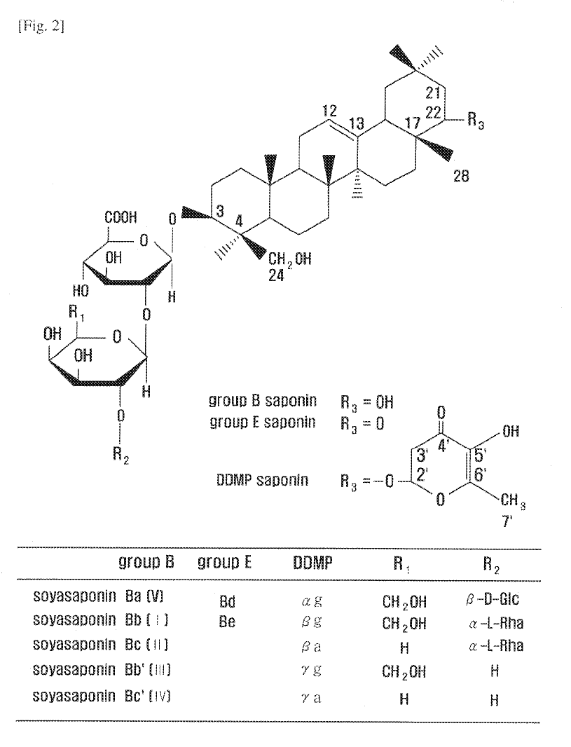

In addition, depending on the scholar soyasaponin compounds corresponding to the group A soyasaponin may be classified as soyasaponins Aa, Ab, Ad, Ae, Af and the like, or may be classified as A1 to A6. Moreover, soyasaponin compounds corresponding to the group B soyasaponin in which no DDMP is bound may be classified as soyasaponins Ba, Bb, Bb′, Bc and the like, or may be classified as soyasaponin I to V. Recently, as new soyasaponin compounds have been identified, the nomenclature has been expressed a little differently. FIG. 1 shows the structure of the group A soyasaponin, FIG. 2 shows the group B soyasaponin, group E soyasaponin, and group DDMP soyasaponin. The soyasaponins shown in FIG. 1 and FIG. 2 can be summarized as shown in Table 1 below.

TABLE 1

| | Name of | | |
|---|---|---|---|
| | soyasaponin | Molecular structure of | Molecular |
| Group soyasaponin | compound | soyasaponin | weight |
| Group A | soyasaponin | glc(1→2)gal(1→2)glcUA(1→3)- | 1364 |
| | Aa(A4) | A- | |
| | | (22←1)ara(3←1)xyl(2,3,4- | |
| | | tri-O-acetyl) | |
| | soyasaponin | glc(1→2)gal(1→2)glcUA(1→3)- | 1436 |
| | Ab(A1) | A- | |
| | | (22←1)ara(3←1)glc(2,3,4,6- | |
| | | tetra-O-acetyl) | |
| | soyasaponin Ac | rha(1→2)gal(1→2)glcUA(1→3)- | 1420 |
| | | A- | |
| | | (22←1)ara(3←1)glc(2,3,4,6- | |
| | | tetra-O-acetyl) | |
| | soyasaponin Ad | glc(1→2)ara(1→2)glcUA(1→3)- | 1390 |
| | | A- | |
| | | (22←1)ara(3←1)glc(2,3,4,6- | |
| | | tetra-O-acetyl) | |
| | soyasaponin | gal(1→2)glcUA(1→3)-A- | 1202 |
| | Ae(A5) | (22←1)ara(3←1)xyl(2,3,4- | |
| | | tri-O-acetyl) | |
| | soyasaponin | gal(1→2)glcUA(1→3)-A- | 1274 |
| | Af(A2) | (22←1)ara(3←1)glc(2,3,4,6- | |
| | | tetra-O-acetyl) | |
| | soyasaponin | ara(1→2)glcUA(1→3)-A- | 1172 |
| | Ag(A6) | (22←1)ara(3←1)xyl(2,3,4- | |
| | | tri-O-acetyl) | |
| | soyasaponin | ara(1→2)glcUA(1→3)-A- | 1244 |
| | Ah(A3) | (22←1)ara(3←1)glc(2,3,4,6- | |
| | | tetra-O-acetyl) | |
| Group B | soyasaponin | glc(1→2)gal(1→2)glcUA(1→3)-B | 958 |
| | Ba(V) | | |
| | soyasaponin Bb | rha(1→2)gal(1→2)glcUA(1→3)-B | 942 |
| | (I) | | |
| | soyasaponin | rha(1→2)ara(1→2)glcUA(1→3)-B | 912 |
| | Bc(II) | | |
| | soyasaponin | gal(1→2)glcUA(1→3)-B | 796 |
| | Bb′(III) | | |
| | soyasaponin | ara(1→2)glcUA(1→3)-B | 766 |
| | Bc′(IV) | | |
| Group | soyasaponin αg | glc(1→2)gal(1→2)glcUA(1→3)- | 1084 |
| DDMP | | B-(22←2′)-DDMP | |
| | soyasaponin βg | rha(1→2)gal(1→2)glcUA(1→3)- | 1068 |
| | | B-(22←2′)-DDMP | |
| | soyasaponin βa | rha(1→2)ara(1→2)glcUA(1→3)- | 1038 |
| | | B-(22←2′)-DDMP | |
| | soyasaponin γg | gal(1→2)glcUA(1→3)-B- | 922 |
| | | (22←2′)-DDMP | |
| | soyasaponin γa | ara(1→2)glcUA(1→3)-B- | 892 |
| | | (22←2′)-DDMP | |
| Group E | soyasaponin Bd | glc(1→2)gal(1→2)glcUA(1→3)-E | 956 |
| | soyasaponin Be | rha(1→2)gal(1→2)glcUA(1→3)-E | 940 |

*“A” represents soyasapogenol A, “B” represents soyasapogenol B, “E” represents soyasapogenol E, “glc” represents β-D-glucopyranosyl, “gal” represents β-D-galactopyranosyl, “glcUA” represents β-D-glucuronopyranosyl, “ara” represents α-Larabinopyranosyl, “rha” represents α-L-rhamnopyranosyl, “xyl” represents β-D-xylopyranosyl, and “DDMP” represents 2,3-dihydro-2,5-dihydroxy-6-methyl-4H-pyran-4-one.

As used herein, the “derivative of soyasaponin” refers to a chemically modified soyasaponin, which means that a substituent has been modified using soyasaponin produced either by isolation from nature or by synthesis.

In the present invention, the soyasaponin can be prepared by isolation from natural materials or by a chemical synthesis method known in the art, and any soyasaponin compounds commercially available can be used.

The composition of the present invention contains, as an active ingredient, soyasaponin, preferably, soyasaponin A or soyasaponin B, more preferably, soyasaponin A, and most preferably, soyasaponin Aa.

The composition of the present invention may contain soyasaponin or a derivative thereof in an amount of 0.001% to 50% by weight, preferably 0.01% to 30% by weight, more preferably 0.1% to 10% by weight, based on the total weight of the composition. If the content is less than 0.001% by weight, the efficacy and effect due to the component are insufficient, and if the content exceeds 50% by weight, there is a problem in skin safety or formulation.

As the composition according to the present invention contains soyasaponin, it can inhibit melanin production and skin pigmentation, thereby providing an excellent skin whitening effect.

The composition according to the present invention, for example, can be formulated into a pharmaceutical composition, a cosmetic composition, an external use skin preparation composition, a health food composition, or oral composition.

The pharmaceutical composition according to the present invention may further contain a pharmaceutical adjuvant such as a preservative, a stabilizer, a hydrating agent or an emulsifying accelerator, a salt and/or a buffer for controlling osmotic pressure, etc., and other therapeutically useful substances, and may be prepared into various formulations for oral or parenteral administration in accordance with a conventional method.

The formulation for oral administration may include, for example, tablet, pill, hard or soft capsule, liquid, suspension, emulsion, syrup, powder, discutient, fine granule, granule, pellet, or the like. These formulations may include, in addition to the active ingredient, a surfactant, a diluent (e.g., lactose, dextrose, sucrose, mannitol, sorbitol, cellulose and glycine), and a lubricant (e.g., silica, talc, stearic acid and magnesium or calcium salt thereof, and polyethylene glycol). The tablet may include a binder such as magnesium aluminum silicate, starch paste, gelatin, tragacanth, methyl cellulose, sodium carboxymethyl cellulose and polyvinylpyrrolidone, and may optionally include a pharmaceutical additive such as a disintegrant including starch, agar, alginic acid or a sodium salt thereof, an absorbent, a colorant, a flavor, a sweetener, or the like. The tablet may be prepared according to a conventional mixing, granulation or coating method. Further, the formulation for parenteral administration may include, for example, injection, drops, ointment, lotion, gel, cream, spray, suspension, emulsion, suppository, patch, etc., but is not limited thereto.

The pharmaceutical composition according to the present invention can be administered via parenteral, rectal, topical, transdermal, subcutaneous routes or the like. The pharmaceutical composition according to the present invention can be, for example, administered topically to the skin.

Determination of the dose of the active ingredient is within the level of those skilled in the art, and the daily dose of a drug will vary depending on various factors, such as the progression of symptoms of a subject, time of onset, age, health condition, complications and the like. For adults, the composition can be typically administered at a dose of 1 μg/kg to 200 mg/kg, preferably, 50 μg/kg to 50 mg/kg by a split-dose method of once to thrice each day, and the dose is not intended to limit the scope of the present invention in any way.

The composition according to the present invention may be a cosmetic composition. The external form of the cosmetic composition contains a cosmetically or dermatologically acceptable medium or base. It may be in any form suitable for topical application, for example, it may be provided in the form of solutions, gels, solids, paste anhydrous products, emulsions obtained by dispersing oil phase in aqueous phase, suspensions, microemulsions, microcapsules, microgranules, or ionic (liposomes) and non-ionic vesicle dispersants, or in the form of cream, skin toner, lotion, powder, ointment, spray or conceal stick. These compositions may be prepared according to a conventional method in the art. The composition according to the present invention can be used in the form of foam or as an aerosol composition further containing a compressed propellant.

When the formulation of the present invention is paste, cream, or gel, animal fiber, vegetable fiber, wax, paraffin, starch, tragacanth, cellulose derivatives, polyethylene glycol, silicon, bentonite, silica, talc, or zinc oxide, etc., can be used as the carrier ingredient.

When the formulation of the present invention is powder or spray, lactose, talc, silica, aluminum hydroxide, calcium silicate, or polyamide powder can be used as the carrier component, and particularly, in the case of spray, it may further include a propellant, such as chlorofluorohydrocarbon, propane/butane or dimethyl ester.

When the formulation of the present invention is a solution or an emulsion, solvents, solvating agents, or emulsifying agents are used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic ester, polyethylene glycol, or sorbitan fatty acid ester.

When the formulation of the present invention is a suspension, liquid diluting agents, such as water, ethanol, or propylene glycol, suspending agents, such as ethoxylated isostearyl alcohol, polyoxyethylene sorbitol ester, and polyoxyethylene sorbitan ester, and microcrystalline cellulose, aluminum meta-hydroxide, bentonite, agar, or tragacanth, etc., can be used as the carrier ingredient.

The composition of the present invention may further contain, in addition to the above-described components, functional additives and components contained in a general skin whitening composition. The functional additives may include components selected from the group consisting of water-soluble vitamins, oil-soluble vitamins, high molecular weight peptides, high molecular weight saccharides, sphingolipids and seaweeds extracts.

The cosmetic composition of the present invention may further be mixed with components contained in general cosmetic compositions, in addition to the above functional additives, if necessary. The mixing components which can be added additionally may include oil and fat components, humectants, emollient agents, surfactants, organic or inorganic pigments, organic powder, UV absorbents, preservatives, bactericides, antioxidants, vegetable extracts, pH adjusting agents, alcohols, coloring agents, flavoring agents, blood circulation promoters, cooling agents, adiaphoretics, purified water, or the like.

Further, the present invention relates to an external use skin preparation including the composition, and the external use skin preparation is a generic term that may include any substances applied to the skin exterior, and cosmetics and medicines of various formulations may be included therein.

In addition, the composition of the present invention may contain a skin absorption promoting substance to increase the effects of increasing skin whitening effect.

### DETAILED DESCRIPTION OF THE EMBODIMENTS

Hereinafter, the constitution and effects of the present invention will be described in more detail by way of Experimental Examples and Preparation Examples. However, these Experimental Examples and Preparation Examples are given for illustrative purposes only, and the scope of the invention is not intended to be limited by these Examples

### [Reference Example 1] Preparation of Soyasaponin

In order to test the efficacy of the composition of the present invention, soyasaponin Aa was purchased from Chengdu Biopurity Phytochemicals Ltd., and used in the test.

### [Test Example 1] Evaluation of the Degree of Melanin Production Inhibition

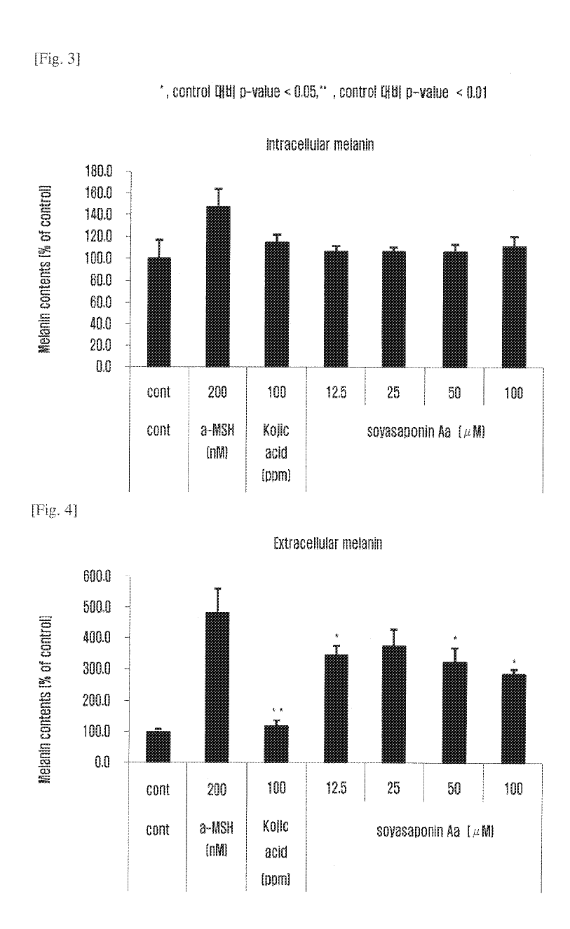

Murine melanoma (B16) cells purchased from the Korean Cell Line Bank were seeded into each well of a 12-well plate (5×10⁴ cells/well) in DMEM (Dulbecco's modified eagle's medium) containing FBS (fetal calf serum) or a medium having growth rates equivalent to or higher than those in the DMEM. The cells were cultured at 37° C. with 5% CO₂ until they reached about 80% confluency. After cultivation, the medium was removed and samples were replaced in medium diluted with 12.5, 25, 50 or 100 μM of soyasaponin Aa, or 100 ppm (corresponding to 700 μM) of kojic acid, and then cultured at 37° C. with 5% CO₂ for 3 days. At this time, α-MSH (melanin stimulation hormone), which is a substance that allows B16 cells to better form melanin during cultivation, was included together in the medium of the other treatment groups except for the untreated control group. In addition, in order to compare the efficacies during the treatment of soyasaponin Aa or kojic acid, the B16 cells were cultured even in a medium containing only α-MSH without containing soyasaponin Aa or kojic acid. The concentration range of the samples was determined through toxicity tests. After removing the medium, the cells were washed with PBS (phosphate buffer saline), and 100 μl of 1M NaOH containing 10% DMSO or cell lysis buffer was added thereto to obtain intracellular melanin in a thermostat at 60° C. The obtained solution was measured for the absorbance at 490 nm using an ELISA reader (Synergy 2, BioTek. USA), and the amount of protein was determined by the Bradford assay, and then the amount of melanin per constant number of cells or the amount of melanin per constant amount of protein was estimated. Melanin production activities (%) of soyasaponin Aa and kojic acid at various concentrations as compared to the control not treated with the sample are shown in FIG. 3 (the degree of intracellular melanin synthesis) and FIG. 4 (the degree of extracellular melanin synthesis).

As shown in FIG. 3 and FIG. 4, it was confirmed that soyasaponin Aa effectively inhibited the synthesis of melanin.

### [Test Example 2] Evaluation of Inhibitory Effect on Tyrosinase Activity

Tyrosinase is an important enzyme that regulates the first step that is performed to produce melanin in the process of pigmentation, and tyrosinase inhibitory activity is known to be an important efficacy mechanism for whitening. Therefore, in this test example, the ability of soyasaponin to inhibit the activity of tyrosinase was evaluated using a method for inhibiting the activity of cell extract tyrosinase. The method for evaluating the cell extract tyrosinase inhibitory activity is based on a phenomenon that melanocyte-derived tyrosinase causes a colorimeteric reaction by allowing substrate DOPA (3,4-dihydroxyphenylalanine; colorless) to form dopacrome (reddish brown).

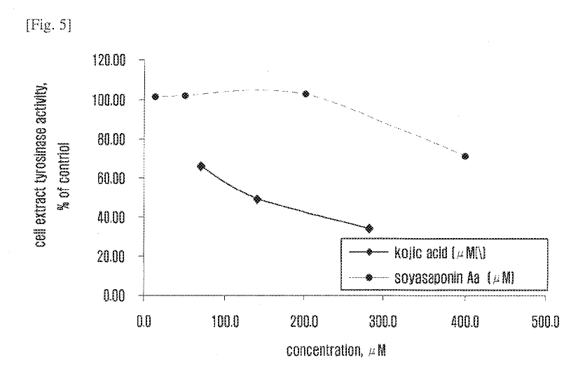

Specific experimental methods are as follows. Kojic acid as a positive control and soyasaponin Aa as an evaluation material were prepared by concentrating four times the concentration to be evaluated. Then, the reaction mixture (pH 6.8, 0.1M potassium phosphate buffer, melan-a cell extract mixture) was prepared in a 96 well plate. Since there may be colors depending on the evaluation material, a reaction mixture containing no cell extract liquid was made into a vehicle for background removal. The plate containing the reaction mixture was covered using a lid or a sheet and incubated at 37° C. for 1 hour. Then, substrate DOPA (2 mg/ml) was added to each well in an amount of 100 μl, and the plate was covered with a lid or a sheet, followed by reaction at 37° C. for 10 minutes. Absorbance was measured at 475 nm using an ELISA reader (Synergy2, BioTek, USA) at intervals of 10 minutes until sufficient colorimeteric reaction had occurred. The results are shown in FIG. 5.

As shown in FIG. 5, the activity of tyrosinase was effectively inhibited after the treatment of soyasaponin Aa.

### [Formulation Example 1] Cosmetic Water

Cosmetic Water was prepared according to a conventional method with the composition shown in Table 2 below.

TABLE 2

| | Content (wt %) |
|---|---|
| Soyasaponin Aa | 2.0 |
| Glycerin | 3.0 |
| Butylene glycol | 2.0 |
| Propylene glycol | 2.0 |
| Carboxyvinyl polymer | 0.1 |
| PEG 12 nonylphenyl ether | 0.2 |
| Polysolvate 80 | 0.4 |
| Ethanol | 10.0 |
| Triethanol amine | 0.1 |
| Preservative, pigment, | Adequate |
| flavoring agent | |
| Purified water | Residual |

### [Formulation Example 2] Nutritive Cream

Nutritive cream was prepared according to a conventional method with the composition shown in Table 3 below.

TABLE 3

| | Content (wt %) |
|---|---|
| Soyasaponin Aa | 2.0 |
| Polysorvate 60 | 1.5 |
| Sorbitan sesquioleate | 0.5 |
| PEG 60 hardened castor oil | 2.0 |
| Liquid paraffin | 10.0 |
| Squalane | 5.0 |
| Caprylic/capric | 5.0 |
| triglyceride | |
| Glycerin | 5.0 |
| Butylene glycol | 3.0 |
| Propylene glycol | 3.0 |
| Triethanol amine | 0.2 |
| Preservative, pigment, | Adequate |
| flavoring agent | |
| Purified water | Residual |

### [Formulation Example 3] Massage Cream

Massage cream was prepared according to a conventional method with the composition shown in Table 4 below.

TABLE 4

| | Content (wt %) |
|---|---|
| Soyasaponin Aa | 1.0 |
| Bees wax | 10.0 |
| Polysorvate 60 | 1.5 |
| PEG 60 hardened castor oil | 2.0 |
| Sorbitan sesquioleate | 0.8 |
| Liquid paraffin | 40.0 |
| Squalane | 5.0 |
| Caprylic/capric | 4.0 |
| triglyceride | |
| Glycerin | 5.0 |
| Butylene glycol | 3.0 |
| Propylene glycol | 3.0 |
| Triethanol amine | 0.2 |
| Preservative, pigment, | Adequate |
| flavoring agent | |
| Purified water | Residual |

### [Formulation Example 4] Pack

Pack was prepared according to a conventional method with the composition shown in Table 5 below.

TABLE 5

| | Content (wt %) |
|---|---|
| Soyasaponin Aa | 10.0 |
| Polyvinyl alcohol | 13.0 |
| Sodium carboxymethyl | 0.2 |
| cellulose | |
| Glycerin | 5.0 |
| Allantoin | 0.1 |
| Ethanol | 6.0 |
| PEG 12 nonylphenyl ether | 0.3 |
| Polysorvate 60 | 0.3 |
| Preservative, pigment, | Adequate |
| flavoring agent | |
| Purified water | Residual |

### [Formulation Example 5] Gel

Gel was prepared according to a conventional method with the composition shown in Table 6 below.

TABLE 6

| | Content (wt %) |
|---|---|
| Soyasaponin Aa | 0.5 |
| Ethylene diamine sodium | 0.05 |
| acetate | |
| Glycerin | 5.0 |
| Carboxy vinyl polymer | 0.3 |
| Ethanol | 5.0 |
| PEG 60 hardened castor oil | 0.5 |
| Triethanol amine | 0.3 |
| Preservative, pigment, | Adequate |
| flavoring agent | |
| Purified water | Residual |

### [Formulation Example 6] Ointment

Ointment was prepared according to a conventional method with the composition shown in Table 7 below.

TABLE 7

| | Content (wt %) |
|---|---|
| Soyasaponin Aa | 0.1 |
| Glycerin | 8.0 |
| Butylene glycol | 4.0 |
| Liquid paraffin | 15.0 |
| Beta glucan | 7.0 |
| Carbomer | 0.1 |
| Caprylic/capric | 3.0 |
| triglyceride | |
| Squalane | 1.0 |
| Cetearyl glucoside | 1.5 |
| Sorbitan stearate | 0.4 |
| Cetearyl glucoside | 1.0 |
| Bees wax | 4.0 |
| Preservative, pigment, | Adequate |
| flavoring agent | |
| Purified water | Residual |

Although specific parts of the present invention have been described in detail, it will be apparent to those skilled in the art that these specific techniques are merely a preferred embodiment and that the scope of the present invention is not limited thereto. Therefore, the substantial scope of the present invention will be defined by the accompanying claims and their equivalents.

1. An external use skin preparation composition for skin-whitening comprising soyasaponin or a derivative thereof as an active ingredient.

2. The composition of claim 1 wherein the soyasaponin is soyasaponin A.

3. The composition of claim 2, wherein the soyasaponin is soyasaponin Aa.

4. The composition of claim 1, wherein the soyasaponin or a derivative thereof is contained in an amount of 0.001% to 50% by weight based on the total weight of the composition.

5. The composition for claim 1, wherein the composition is for improving complexion and skin tone.

6. The composition of claim 1, wherein the composition is a cosmetic composition.

7. A cosmetic for skin whitening comprising the composition of claim 1.

8. The cosmetic of claim 7, wherein the cosmetic is formulated in the form of cream, skin, lotion, powder, ointment, spray or conceal stick.

9. Use of soyasaponin or a derivative thereof as a skin-whitening agent in the preparation of an external use skin preparation composition.
